(19) United States
(12) Patent Application Publication
(10) Pub. No.: US 2002/0016359 A1
(43) Pub. Date: Feb. 7, 2002

(54) Compositions and methods of treating neurodegenerative diseases

(76) Inventors: Mark R. Hellberg, Arlington, TX (US); Jon C. Nixon, Belhaven, NC (US); Billie M. York JR., Conroe, TX (US)

Correspondence Address: ALCON RESEARCH, LTD., R&D COUNSEL, Q-148, 6201 SOUTH FREEWAY, FORT WORTH, TX, 76134-2099, US

(21) Appl. No.: 09/862,132

(22) Filed: May 21, 2001

Related U.S. Application Data
(60) Provisional application No. 60/214,902, filed on Jun. 29, 2000.

(51) Int. Cl. A61K 31/352; A61K 31/343; A61K 31/216

(52) U.S. Cl. 514/456; 514/469; 514/533; 514/534

(57) ABSTRACT

Methods for treating neurodegenerative diseases and disorders are disclosed. The methods utilize compositions containing certain compounds having an anti-inflammatory and anti-oxidant moiety covalently linked by an amide or ester bond.

### CLAIM FOR PRIORITY

Pursuant to 35 U.S.C. §120, Applicants hereby claim priority based on Provisional Application Serial No. 60/214,902

### BACKGROUND OF THE INVENTION

The present invention relates to the treatment of neurodegenerative diseases. In particular, the present invention is directed to the prevention, treatment or amelioration of neurodegenerative diseases by administration to a subject certain bi-functional compounds.

The primary causes of neurogenerative diseases such as Alzheimer's disease, Parkinson's disease, muscular sclerosis, amyotropic lateral sclerosis are far from being fully elucidated. In general, neurodegenerative diseases can be characterized by a loss of transmitter specific neurons. These conditions generally progress and ultimately impact the function of several neuronal systems over the course of the disease. To date, no single specific mechanism can explain the heterogeneous family of disorders that results in neurodegeneration. Several factors including oxidative stress and inflammation do appear to play an important role in the etiology of these disorders. (See, Nature, volume 382, pages 120-1 (1996), Pyschopharmacology Bulletin, volume 32, pages 343-52 (1996), Arzneimittelforschung, volume 45, pages 443-6 (1995)).

Oxidative stress has been increasingly implicated in a number of neurodegenerative diseases. Among these conditions are Alzheimer's disease, Parkinson's disease, muscular sclerosis, amyotropic lateral sclerosis (Brain Research, volume 696, pages 268-71 (1996)). Additionally, the observed upregulation of heme-oxidase-1 in Alzheimer's patients supports the suggestion that affected brain tissues are experiencing chronic oxidative stress (Annals of Neurology, volume 37, pages 758-86 (1995), American Journal of Pathology, volume 145, pages 42-7 (1994), and FASEB Journal, volume 10, pages 709-720 (1996)).

Likewise pathways that are mediated by inflammatory processes have also been implicated in neurodegeneration. Mechanisms such as abnormal filament formation can either directly or indirectly result in the activation of the resident microglia population. Microglia activation leads to chemokine and cytokine expression, cell proliferation and chemotaxis (Biochemical and Biophysics Research Communications, volume 225, pages 474-8 (1996), and Proceedings of the National Academy of Science U.S.A., volume 92, pages 10738-41 (1995)). This activation of inflammatory processes plays an important role generating the changes associated with neurodegeneration. (See, Proceedings of the National Academy of Science U.S.A., volume 92, pages 3032-5 (1995), and Proceedings of the National Academy of Science U.S.A., volume 94, pages 5296-5301 (1997)).

Antioxidants and nonsteroidal anti-inflammatory agents have demonstrated efficacy in slowing the progression of Alzheimer's disease. A recent study suggests that the use of alpha tocopherol (vitamin E) and/or selegiline may delay clinically important functional deterioration in patients with Alzheimer's disease (New England Journal of Medicine, volume 336, pages 1216-22 (1997)). Although the beneficial effect of alpha tocopherol is related to its activity as a lipid soluble antioxidant, the mechanism of the selegiline effect is open to speculation. While selegiline is a selective and irreversible mono amine oxidase B inhibitor, it has the capacity to increase the level of several antioxidant enzymes (Life Science, volume 59, pages 1047-53 (1996), and Annals of the New York Academy of Science, volume 786, pages 391-409 (1996)). The combination of the two compounds, however, did not result in a further decrease in the rate of functional deterioration which suggests that these compounds share a common mechanism.

It has also been reported that the symptoms of Alzheimer's disease are attenuated by the administration of non-steroidal anti-inflammatory drugs. (Neurobiology Aging, volume 17, pages 789-94 (1996), and Annual Review of Medicine, volume 47, pages 401-11 (1996)).

U.S. Pat. No. 5,607,966 (Hellberg et al.), U.S. Pat. No. 5,643,943 (Gamache et al.), U.S. Pat. No. 5,811,438 (Hellberg et al.) and U.S. Pat. No. 5,811,453 (Yanni et al.) disclose bi-functional compounds, compositions and methods of use for the treatment of ocular inflammation and cardiovascular diseases. The preceding patents, however, do not disclose the use of the compounds for treating brain or spinal column diseases and disorders.

### SUMMARY OF INVENTION

The present invention is directed to compositions comprising certain bi-functional compounds and methods of treating neurodegenerative diseases and disorders.

The bi-functional compounds of the present invention include both a non-steroidal anti-inflammatory agent (“NSAIA”) moiety and an anti-oxidant moiety. The compounds possess certain features believed to be useful in providing therapy of neurodegenerative diseases and disorders. The lipophilic nature of these compounds is believed to promote penetration into the brain and other neurological tissues. Other properties of the compounds useful in methods of the present invention include the inhibition of cellular adhesion, proliferation, activation of transcription factors and cytokine expression. By reducing oxidative stress, suppressing microglial activation, and reducing prostaglandin-mediated inflammatory processes these compounds provide a multiple mechanism approach to treating neurodegenerative diseases.

The methods of the present invention are directed to the prevention, treatment or amelioration of neurodegenerative diseases including, but not limited to, Alzheimer's disease, Parkinson's disease, muscular sclerosis, amyotropic lateral sclerosis, Huntington's disease and diseases mediated by glutamate toxicity.

### DETAILED DESCRIPTION OF INVENTION

The bi-functional compounds useful in the methods of the present invention are of the formula (I):

A—X—(CH₂)ₙ—Y—(CH₂)ₘ—Z (I)

wherein:

A is an non-steroidal anti-inflammatory agent (NSAIA) originally having a carboxylic acid;

A—X is an ester or amide linkage derived from the carboxylic acid moiety of the NSAIA, wherein X is O or NR;

R is H, C₁-C₆ alkyl or C₃-C₆ cycloalkyl;

Y, if present, is O, NR, C(R)₂, CH(OH) or S(O)_{n′};

n is 2 to 4 and m is 1 to 4 when Y is O, NR, or S(O)_{n′};

n is 0 to 4 and m is 0 to 4 when Y is C(R)₂ or is not present;

n is 1 to 4 and m is 0 to 4 when Y is CH(OH);

n′ is 0 to 2; and

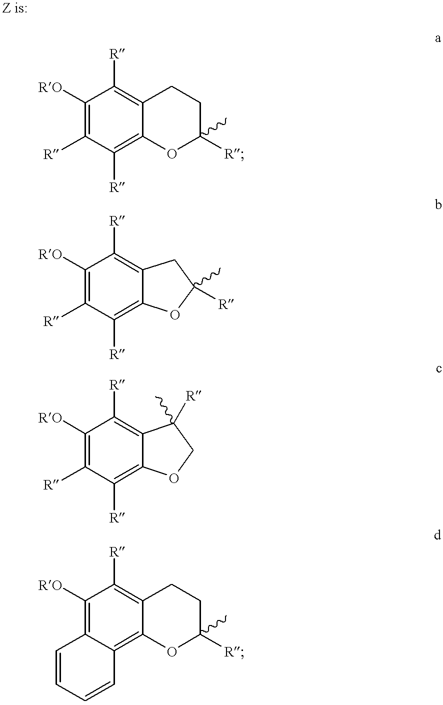

wherein:

R′ is H, C(O)R, C(O)N(R)₂, PO₃⁻, or SO₃⁻; and

R″ is H or C₁-C₆ alkyl.

The bi-functional compounds of the present invention also include various stereoisomers or racemic mixtures of any of the compounds contemplated within formula (I), and pharmaceutically acceptable salts of the compounds of formula (I).

The bi-functional compounds of the present invention contain a non-steroidal anti-inflammatory agent, “A”, originally having a carboxylic acid moiety. A number of chemical classes of non-steroidal anti-inflammatory agents have been identified. The following text, the entire contents of which are incorporated herein by reference to the extent it refers to NSAIAs having a carboxylic acid, may be referred to for various NSAIA chemical classes: CRC Handbook of Eicosanoids: Prostaglandins, and Related Lipids, Volume II, Drugs Acting Via the Eicosanoids, pages 59-133, CRC Press, Boca Raton, Fla. (1989). The NSAIA may be selected, therefore, from a variety of chemical classes including, but not limited to, fenamic acids, such as flufenamic acid, niflumic acid and mefenamic acid; indoles, such as indomethacin, sulindac and tolmetin; phenylalkanoic acids, such as suprofen, ketorolac, flurbiprofen, ibuprofen and diclofenac. Further examples of NSAIAs are listed below:

| | | |
|---|---|---|
| loxoprofen | tolfenamic acid | indoprofen |
| pirprofen | Clidanac | fenoprofen |
| naproxen | Fenclorac | meclofenamate |
| benoxaprofen | Carprofen | isofezolac |
| aceloferac | Fenbufen | etodolic acid |
| fleclozic acid | Amfenac | efenamic acid |
| bromfenac | Ketoprofen | fenclofenac |
| alcofenac | Orpanoxin | zomopirac |
| diflunisal | pranoprofen | zaltoprofen |

The preferred compounds of formula (I) are those wherein “A” is selected from the ester or amide derivatives of naproxen, flurbiprofen or diclofenac. The most preferred compounds are those wherein “A” is selected from the ester or amide derivatives of naproxen or flurbiprofen.

With respect to the other substituents of the compounds of formula (I), the preferred compounds are those wherein:

X is O or NR;

R is H or C₁ alkyl;

Y is CH(OH), and m is 0 to 2 and n is 1 or 2, or Y is not present, and m is 1 or 2 and n is 0 to 4;

Z is a, b or d;

R′ is H or C(O)CH hd 3; and

R″ is CH₃.

The most preferred compounds are those wherein:

X is O or NR;

R is H;

Y is not present;

m is 0 or 1;

n is 1;

Z is a, or b;

R′ is H; C(O)CH₃; and

R″ is CH₃.

The following compounds are particularly preferred:

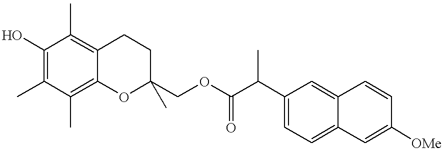

2-(6-hydroxy-2,5,7,8-tetramethyl-3,4-dihydro-2H-benzo[1,2-b]pyran-2-yl)methyl 2-(6-methoxy-2-naphthyl)propionate (“Compound A”)

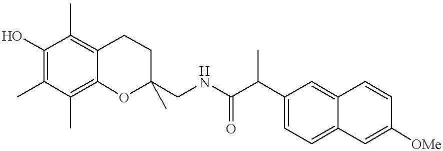

N-(2-(6-hydroxy-2,5,7,8-tetramethyl-3,4-dihydro-2H-benzo[1,2-b]pyran-2-yl)methyl) 2-(6-methoxy-2-naphthyl)propionamide (“Compound B”)

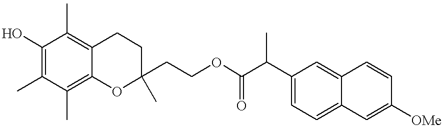

2-(6-hydroxy-2,5,7,8-tetramethyl-3,4-dihydro-2H-benzo[1,2-b]pyran-2-yl)ethyl 2-(6-methoxy-2-naphthyl)propionate (“Compound C”)

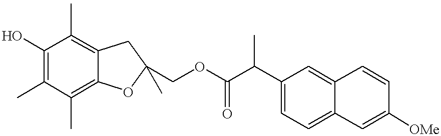

2-(5-hydroxy-2,4,6,7-tetramethyl-2,3-dihydro-benzo[1,2-b]furan-2-yl)methyl 2-(6-methoxy-2-naphthyl)propionate (“Compound D”)

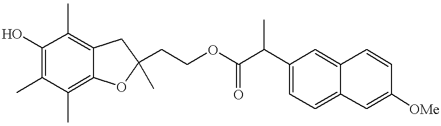

2-(5-hydroxy-2,4,6,7-tetramethyl-2,3-dihydro-benzo[1,2-b]furan-2-yl)ethyl 2-(6-methoxy-2-naphthyl)propionate (“Compound E”); and

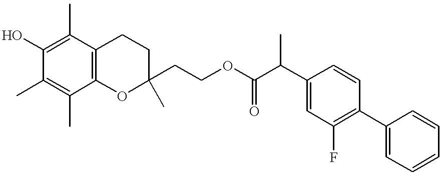

2-(6-hydroxy-2,5,7,8-tetramethyl-2,3-dihydro-2H-benzo[1,2-b]pyran-2-yl)ethyl (2-(3-fluoro-4-phenyl-phenyl)propionate (“Compound F”).

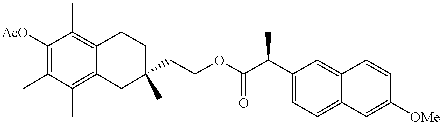

(S)-6-methoxy-α-methyl-naphthaleneacetic acid, (R)-2-(6-acetoxy-3,4-dihydro-2,5,7,8-tetramethyl-2H-1-benzopyran-2-yl)ethyl ester (“Compound G”),

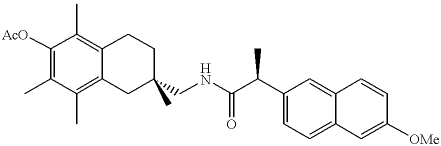

(R)N-(2-(6-acetoxy-2,5,7,8-tetramethyl-3,4-dihydro-2H-benzo[1,2-b]pyran-2-yl)methyl) (S) (2-(6-methoxy-2-naphthyl)propionamide (“Compound H”);

The most preferred bifunctional compound of the present invention is:

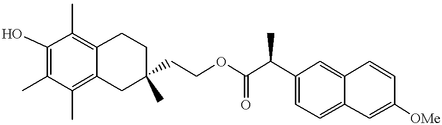

(S)-6-methoxy-α-methyl-naphthaleneacetic acid, (R)-2-(3,4-dihydro-6-hydroxy-2,5,7,8-tetramethyl -2H-1-benzopyran-2-yl)ethyl ester (“Compound X”), which is a particular stereoisomer of Compound C.

The compounds of formula (I) may be prepared by methods disclosed in U.S. Pat. No. 5,607,966 (Hellberg et al.), the entire contents of which are incorporated herein by reference.

As stated above, the present invention is directed to methods employing compositions adapted for the prevention, treatment or amelioration of neurological diseases or disorders. Such methods include, but not limited to, head trauma, muscular sclerosis, epilepsy and other seizure disorders, Alzheimer's disease, Parkinson's diseases, Huntington's disease. Also included are methods to treat neurological diseases or disorders where excitotoxic events (e.g., glutamate-mediated excitotoxicity) are known to participate. The methods of the present invention are also directed to the treatment of spinal cord diseases, disorders or injuries, including Lou Gehrig's disease (amyotropic lateral sclerosis, ALS). Also included is the treatment of other diseases of the spinal cord where excitotoxic events are known to participate.

The compositions of the present invention can be administered to the patient prophylactically or during the progression of the disease or disorder. The compositions generally will be administered systemically. Systemic administration will generally involve oral, or parenteral administration.

The compositions of the present invention will include one or more compounds of formula (I) and a pharmaceutically acceptable vehicle for said compound(s). Various excipients may be contained in the tablets, capsules, solutions, suspensions or gels of the present invention. For example, such excipients may include: buffers (e.g., borate, carbonate, phosphate), tonicity agents (e.g., sodium chloride, potassium chloride, polyols), preservatives (e.g., polyquaterniums, polybiguanides, benzalkonium chloride (BAC)), chelating agents (e.g., EDTA), viscosity enhancing agents (e.g., polyethoxylated glycols) and solubilizing agents (e.g., polyethoxylated castor oils, including polyoxl-35 castor oil (Cremophor EL®, BASF Corp., Parsippany, N.J.); Polysorbate 20, 60 and 80; Pluronic® F-68, F-84 and P-103 (BASF Corp.); or cyclodextrin).

When the compounds of formula (I) are administered during intracerebral or intraspinal surgical procedures, such as injection, or intraspinal or intracerebral injection or perfusion, the use of irrigating solutions as vehicles are most preferred. The most basic irrigating solutions generally comprise saline, or phosphate-buffered saline. More advanced irrigating solutions, however, are preferred. As used herein, the term “physiologically balanced irrigating solution” refers to a solution which is adapted to maintain the physical structure and function of tissues during invasive or noninvasive medical procedures. This type of solution will typically contain electrolytes, such as sodium, potassium, calcium, magnesium and/or chloride; an energy source, such as dextrose; and a bicarbonate-buffer to maintain the pH of the solution at or near physiological levels. Various solutions of this type are known (e.g., Lactated Ringers Solution). BSS® Sterile Irrigating Solution and BSS Plus® Sterile Intraocular Irrigating Solution (Alcon Laboratories, Inc., Fort Worth, Tex., USA) are examples of physiologically balanced intraocular irrigating solutions. The latter type of solution is described in U.S. Pat. No. 4,550,022 (Garabedian, et al.), the entire contents of which are incorporated herein by reference.

Systemic compositions of the present invention can be formulated by well known techniques in the art. Oral compositions will generally be in the form of tablets, hard or soft gelatin capsules, suspensions, granules, powders or other typical compositions and will contain excipients typically present in such compositions. Methods for the preparation of such oral vehicles are well known by those skilled in the art. Parenteral administration (e.g., intravenous or intramuscular) compositions will be generally be in the form of injectable solutions or suspensions. Methods for the preparation of such parenteral compositions are well known by those skilled in the art.

In general, the formula (I) concentrations of the compositions and the doses used for the above described purposes will vary, but will be in an effective amount to prevent, reduce or ameliorate neuronal tissue damage. As used herein, “therapeutically effective amount” refers to an amount of at least one compound of formula (I) which will prevent, reduce or ameliorate neuronal tissue damage in a mammal.

Irrigation solutions will generally contain one or more of the compounds of formula (I) in a concentration of from about 0.01 μM to about 100 μM. The volume of irrigation solution administered will depend on the administrative procedure, e.g., intraocular irrigation, or intraocular or intracerebral injection, and the particular condition being treated.

For systemic treatments, the patient will generally be dosed with a compound of formula (I) in an amount of about 0.02 to 10 mg/kg/day in single or divided doses, regardless of the route of administration (i.e., oral, parenteral (s.c., i.m., i.v.)). Systemic treatments involving the oral route of administration are preferred.

Any of the above-described vehicles or other vehicles known in the art may be employed in the compositions of the present invention, provided such vehicles allow for the administration of a compound of formula (I) to the tissue to be treated and do not cause significant side effects to the patient. As used herein, such a vehicle is referred to as a “pharmaceutically acceptable vehicle.”

What is claimed is:

1. A method for the treatment, prevention or amelioration of brain or spinal cord disease, disorder or injury comprising a pharmaceutically acceptable vehicle and an amount of one or more compounds of formula (I):
A—X—(CH₂)ₙ—Y—(CH₂)ₘ—Z (I)
wherein:
A—X is an non-steroidal anti-inflammatory agent (NSAIA) originally having a carboxylic acid;
A—X is an ester or amide linkage derived from the carboxylic acid moiety of the NSAIA, wherein X is O or NR;
R is H, C₁-C₆ alkyl or C₃-C₆ cycloalkyl;
Y, if present, is O, NR, C(R)₂, CH(OH) or S(O)_{n′};
n is 2 to 4 and m is 1 to 4 when Y is O, NR, or S(O)_{n′};
n is 0 to 4 and m is 0 to 4 when Y is C(R)₂ or is not present;
n is 1 to 4 and m is 0 to 4 when Y is CH(OH);
n′ is0 to 2; and
Z is:
wherein: 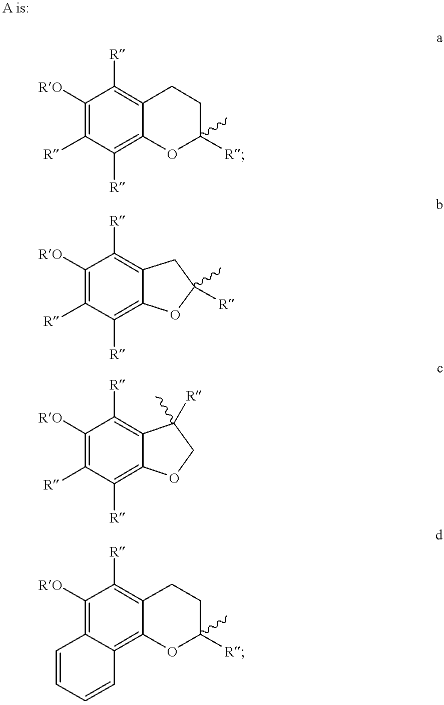
wherein:
R′ is H, C(O)R, C(O)N(R)₂, PO₃⁻, or SO₃⁻; and
R″ is H or C₁-C₆ alkyl; and pharmaceutically acceptable salts therefor.

2. A method according to claim 1, wherein:
X is O or NR;
R is H or C₁ alkyl;
Y is CH(OH), and m is 0 to 2 and n is 1 or 2, or Y is not present, and m is 1 or 2 and n is 0 to 4;
Z is a, b or d;
R′ is H or C(O)CH₃; and
R″ is CH₃.

3. A method according to claim 1, wherein the non-steroidal anti-inflammatory agent is selected from the group consisting of: fenamic acids; indoles; and phenylalkanoic acids.

4. A method according to claim 1, wherein the non-steroidal anti-inflammatory agent is selected from the group consisting of:
loxoprofen; tolfenamic acid; indoprofen; pirprofen; clidanac; fenoprofen; naproxen; fenclorac; meclofenamate; benoxaprofen; carprofen; isofezolac; aceloferac; fenbufen; etodolic acid; fleclozic acid; amfenac; efenamic acid; bromfenac; ketoprofen; fenclofenac; alcofenac; orpanoxin; zomopirac; diflunisal; flufenamic acid; niflumic acid; mefenamic acid; pranoprofen, zaltoprofen; indomethacin; sulindac; tolmetin; suprofen; ketorolac; flurbiprofen; ibuprofen; and diclofenac.

5. A method according to claim 4, wherein:
X is O or NR;
R is H or C₁, alkyl;
Y is CH(OH), and m is 0 to 2 and n is 1 or 2, or Y is not present, and m is 1 or 2 and n is 0 to 4;
Z is a, b or d;
R′ is H or C(O)CH₃; and
R″ is CH₃.

6. A method according to claim 4, wherein the non-steroidal anti-inflammatory agent is selected from the group consisting of naproxen, flurbiprofen and diclofenac.

7. A method according to claim 1, wherein the compound of formula (I) is selected from the group consisting of:

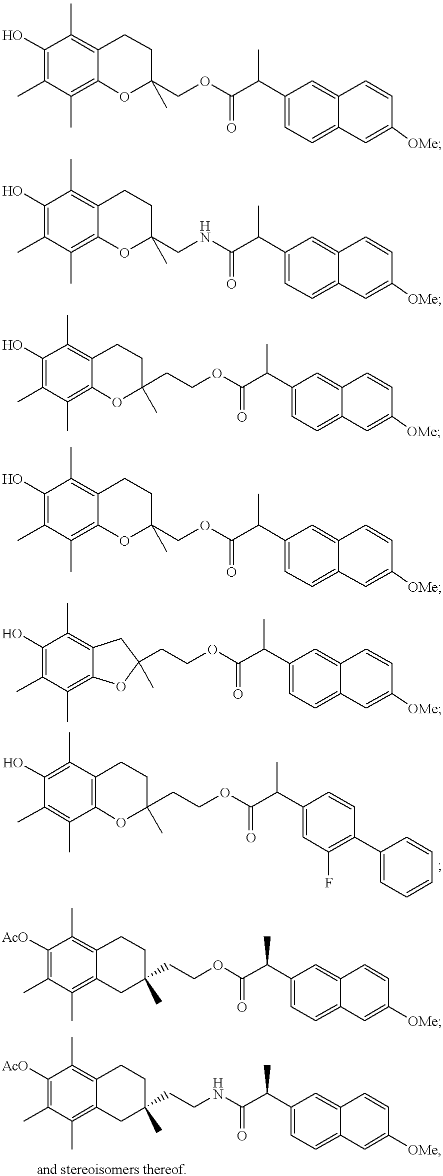

8. A method according to claim 1, wherein the compound of formula (I) is:

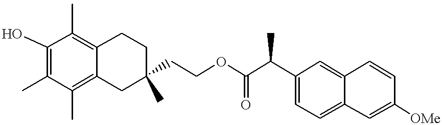

9. A method according to claim 6, wherein:
X is O or NR;
R is H or C₁, alkyl;
Y is CH(OH), and m is 0 to 2 and n is 1 or 2, or Y is not present, and m is 1 or 2 and n is 0 to 4;
Z is a, b or d;
R′ is H or C(O)CH₃; and
R″ is CH₃.

10. A method according to claim 1, wherein the vehicle is/are local or systemic compositions or a physiologically balanced irrigating solution, or a combinations thereof.

11. A method according to claim 4, wherein the vehicle is/are local or systemic compositions or a physiologically balanced irrigating solution, or a combinations thereof.

12. A method according to claim 7, wherein the vehicle is/are local or systemic compositions or a physiologically balanced irrigating solution, or a combinations thereof.

13. A method according to claim 8, wherein the vehicle is/are local or systemic compositions or a physiologically balanced irrigating solution, or a combinations thereof.

14. A method according to claim 1, wherein the brain or spinal cord disease, disorder or injury is head trauma, muscular sclerosis, amyotropic lateral sclerosis, epilepsy and other seizure disorders, Alzheimer's disease, Huntington's disease, and other diseases, disorders or injuries where excitotoxic events are known to participate.
